(19) United States
(12) Patent Application Publication
(10) Pub. No.: US 2025/0090812 A1
(43) Pub. Date: Mar. 20, 2025

(54) ACTIVELY STEERABLE GUIDEWIRE

(71) Applicants: Boston Scientific Scimed, Inc., Maple Grove, MN (US); Boston Scientific Medical Device Limited, Galway (IE)

(72) Inventors: Deepak Kumar Sharma, Muzaffarnagar (IN); James J. Scutti, Norwell, MA (US); Juan Pablo Ortiz Garcia, Heredia (CR); Charles A. Gibson, Malden, MA (US)

(73) Assignees: Boston Scientific Scimed, Inc., Maple Grove, MN (US); Boston Scientific Medical Device Limited, Galway (IE)

(21) Appl. No.: 18/828,233

(22) Filed: Sep. 9, 2024

Related U.S. Application Data
(60) Provisional application No. 63/583,015, filed on Sep. 15, 2023.

(51) Int. Cl. A61M 25/09; A61M 25/01

(52) U.S. Cl. CPC A61M 25/09; A61M 25/0136

(57) ABSTRACT

A steerable elongate medical device includes a composite shaft and a handle that is removably securable to the composite shaft. The composite shaft includes a first shaft component and a second shaft component. The composite shaft bends in a first direction when the first shaft component is heated above a transition temperature and bends in the first direction. The composite shaft bends in an opposing second direction when the second shaft component is heated above the transition temperature and bends in the second direction. The handle enables a user to selectively pass an electrical current through either of the first shaft component or the shaft component in order to electrically heat the selected shaft component.

### CROSS REFERENCE TO RELATED APPLICATIONS

The present application claims the benefit of and priority to U.S. Provisional Patent Application Ser. No. 63/583,015, filed on Sep. 15, 2023, the disclosure of which is incorporated herein by reference.

### TECHNICAL FIELD

The disclosure pertains to medical devices. More particularly, the disclosure pertains to medical devices such as guidewires.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

The disclosure is directed to several alternative designs, materials and methods of manufacturing medical device structures and assemblies, and the use thereof. An example may be found in a steerable elongate medical device. The steerable elongate medical device includes a composite shaft extending from a proximal region to a distal region, the distal region adapted to selectively bend in a first direction or an opposing second direction. The composite shaft includes a first shaft component having a straight configuration below a transition temperature and a curved configuration above the transition temperature, the first shaft component causing the composite shaft to bend in the first direction when the first shaft component is in its curved configuration. The composite shaft includes a second shaft component having a straight configuration below the transition temperature and a curved configuration when above the transition temperature, the second shaft component causing the composite shaft to bend in the second direction when the second shaft component is in its curved configuration. An intervening member separates the first shaft component and the second shaft component. The steerable elongate medical device includes a handle removably secured to the proximal region. The handle includes a housing adapted to be removably secured to the proximal region of the composite shaft and a knob rotatably secured relative to the housing. The knob includes a knob body, a first electrical contact disposed on the knob body and adapted to contact the first shaft component when the knob is rotated to a first position, and a second electrical contact disposed on the knob body and adapted to contact the second shaft component when the knob is rotated to a second position.

Alternatively or additionally, the first shaft component may be adapted to increase in temperature when an electrical current passes through the first shaft component, and the second shaft may be is adapted to increase in temperature when an electrical current passes through the second shaft component.

Alternatively or additionally, the first shaft component and the second shaft component may each include a shape memory metal.

Alternatively or additionally, the first shaft component and the second shaft component each include nitinol.

Alternatively or additionally, the first electrical contact may be adapted to make electrical contact with the first shaft component when the knob is rotated to the first position, and the second electrical contact is adapted to make electrical contact with the second shaft component when the knob is rotated to the second position.

Alternatively or additionally, the knob may be adapted to be rotated to a neutral position in between the first position and the second position in which neither of the first electrical contact or the second electrical contact make electrical contact with either the first shaft component or the second shaft component.

Alternatively or additionally, the knob may further include a power connector that is electrically coupled with the first electrical contact and the second electrical contact.

Alternatively or additionally, the steerable elongate medical device may further include a conductive member in electrical contact with a distal region of the first shaft component and a distal region of the second shaft component in order to complete a circuit.

Alternatively or additionally, the transition temperature may be above 37° C.

Alternatively or additionally, the intervening member may include an insulating member.

Alternatively or additionally, the intervening member may include a thermoelectric circuit adapted to selectively heat one of the first shaft component and the second shaft component.

Alternatively or additionally, the first shaft component may include a plurality of cuts formed within the first shaft component in order to enhance articulation of the first shaft component.

Alternatively or additionally, the second shaft component may include a plurality of cuts formed within the second shaft component in order to enhance articulation of the second shaft component.

Another example may be found in a steerable guidewire. The steerable guidewire includes a composite shaft that is adapted to selectively bend in a first direction or an opposing second direction. The composite shaft includes a first shaft component movable between a first configuration and a second configuration, the first shaft component adapted to move into the second configuration when electrically heated. The composite shaft includes a second shaft component movable between a first configuration and a second configuration, the second shaft component adapted to move into the second configuration when electrically heated. A handle is adapted to be removably secured to the composite shaft. The handle is selectable between a first position in which an electrical current is provided to the first shaft component, a second position in which an electrical current is provided to the second shaft component, and a third position in which no electrical current is provided.

Alternatively or additionally, the first shaft component and the second shaft component may each include a shape memory metal.

Alternatively or additionally, the first shaft component and the second shaft component may each include nitinol.

Alternatively or additionally, the first configuration of the first shaft component and the first configuration of the second shaft component may each correspond to a straight configuration of the composite shaft.

Alternatively or additionally, the first shaft component may cause the composite shaft to bend in the first direction when the first shaft component is electrically heated and the second shaft component is not electrically heated and the second shaft component may cause the composite shaft to bend in the second direction when the second direction is electrically heated and the first shaft component is not electrically heated.

Alternatively or additionally, the composite shaft may have a neutral configuration when neither the first shaft component nor the second shaft component are electrically heated.

Another example may be found in a steerable guidewire. The steerable guidewire includes a composite shaft adapted to selectively bend in a first direction or an opposing second direction. The composite shaft includes a first shaft component movable between a neutral configuration and a curved configuration, the first shaft component adapted to move into the curved configuration when electrically heated, the first shaft component including nitinol. The composite shaft includes a second shaft component movable between a neutral configuration and a curved configuration, the second shaft including adapted to move into the curved configuration when electrically heated, the second shaft component comprising nitinol. The composite shaft is adapted to bend in the first direction when the first shaft component is in its curved configuration and the second shaft component is in its neutral configuration. The composite shaft is adapted to bend in the opposing second direction when the first shaft component is in its neutral configuration and the second shaft component is in its curved configuration. The composite shaft is adapted to remain in a neutral configuration when the first shaft component is in its neutral configuration and the second shaft component is in its neutral configuration.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a perspective of an illustrative steerable medical device;

FIG. 2 is a schematic view of an illustrative composite shaft, showing possible degrees of freedom;

FIG. 3 is a perspective view of the illustrative steerable medical device of FIG. 1, with part of the handle removed;

FIG. 4 is a schematic view of an illustrative composite shaft forming part of the illustrative steerable medical device of FIG. 1, in which an insulating material is assembled between two thermal shape memory materials;

FIG. 5 is a schematic view of the illustrative steerable medical device of FIG. 1, showing the knob in a first position in which a first shaft component of the composite shaft is electrified;

FIG. 6 is a schematic view of the illustrative steerable medical device of FIG. 1, showing the knob in a second position in which a second shaft component of the composite shaft is electrified;

FIG. 7 is schematic view of the illustrative steerable medical device of FIG. 1, showing the knob in a third position in which neither shaft component of the composite shaft is electrified;

FIG. 8 is a cross-sectional view taken along line 8-8 of FIG. 4;

FIG. 9 is a perspective view of the illustrative composite shaft, showing an example cut pattern;

FIG. 10 is a schematic view of an illustrative composite shaft that may be used in forming the illustrative steerable medical device of FIG. 1, in which a thermoelectric circuit is assembled between two thermal shape memory materials; and

FIG. 11 is a schematic view of an illustrative thermoelectric circuit usable within the illustrative composite shaft of FIG. 10.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

A variety of medical procedures utilize guidewires. In many instances, a guidewire may be used to access a portion of the anatomy, such as but not limited to within the vasculature. Once the guidewire has accessed the desired location, other medical devices may be advanced over the guidewire. In some instances, accessing the desired location within the vasculature may entail passing through tortuous passages within the vasculature. In some instances, accessing the desired location within the vasculature may entail successfully advancing the guidewire into and through a side branch of a vessel, which entails finding and penetrating the ostium of the side branch. In some instances, being able to steer the guidewire may be beneficial in successfully navigating tortuous passages within the vasculature as well as successfully advancing within a side branch.

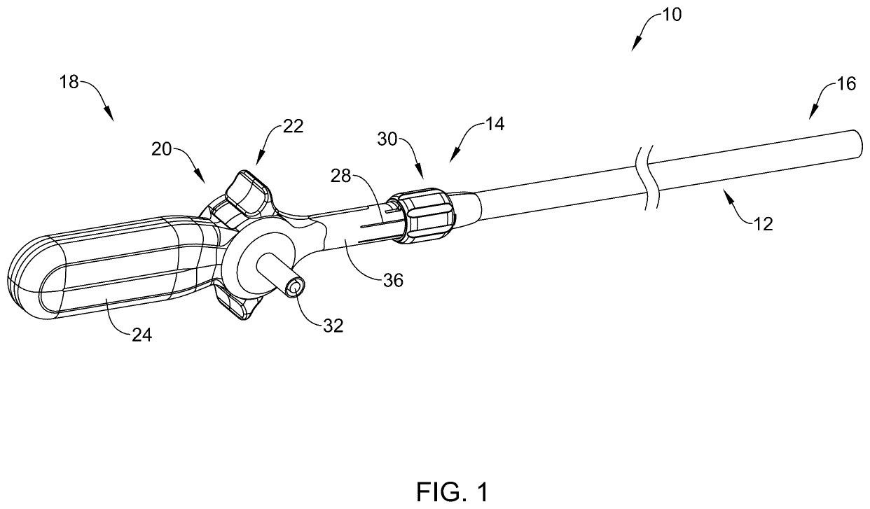

FIG. 1 is a perspective view of an illustrative steerable elongate medical device 10. The steerable elongate medical device 10 may be considered as being a steerable guidewire, for example. The steerable guidewire 10 includes a composite shaft 12 that extends from a proximal region 14 to a distal region 16 as well as a handle 18 that is adapted to be removably secured to the proximal region 14 of the composite shaft 12. It will be appreciated that once the steerable guidewire 10 has successfully reached a desired location such as a treatment site within the vasculature, the handle 18 may be removed so that other medical devices such as therapeutic devices may be advanced over the steerable guidewire 10.

The handle 18 may include a housing 20 that is adapted to be removably secured to the proximal region 14 of the composite shaft 12. The handle 18 may include a knob 22 that is rotatably secured relative to the housing 20. As will be discussed, rotating the knob 22 may enable actuating the composite shaft 12 to bend in a first direction or in a second direction, for example. In some instances, the housing 20 may include a proximal region 24 that is adapted to facilitate grasping the steerable elongate medical device 10. The housing 20 may include a distal region 26 that is adapted to fit over the proximal region 14 of the composite shaft 12. In some instances, the distal region 26 may be narrowed relative to the distal region 26. In some instances, the distal region 26 may include slots 28 formed within the distal region 26 in order to allow the distal region 26 to flex sufficiently to enable insertion (and subsequent removal) of the composite shaft 12 from the handle 18.

In some instances, a collet 30 may be disposed over the distal region 26 and may be used to compress the distal region 26 onto the proximal region 14 of the composite shaft 12. In some instances, the collet 30 may be adapted to be slide distally into the position shown, after the handle 18 has been disposed over the proximal region 14 of the composite shaft 12 in order to secure the handle 18 in position relative to the proximal region 14 of the composite shaft 12 by compressing the distal region 26 onto the proximal region 14 of the composite shaft 12. In some instances, the collet 30 may include a first half and a second half, where each of the first half and the second half have a semi-cylindrical profile and together form a cylindrical profile. The first half and the second half may be adapted to be snapped together on the distal region 26 in order to compress the distal region 26 of the housing 20 onto the proximal region 14 of the composite shaft 12. In some instances, the collet 30 may have a threaded engagement with the distal region 26 of the housing 20. In some instances, the handle 18 may include a power connector 32 that may be used to provide the electrical current that is used to actuate the composite shaft 12 (as will be discussed).

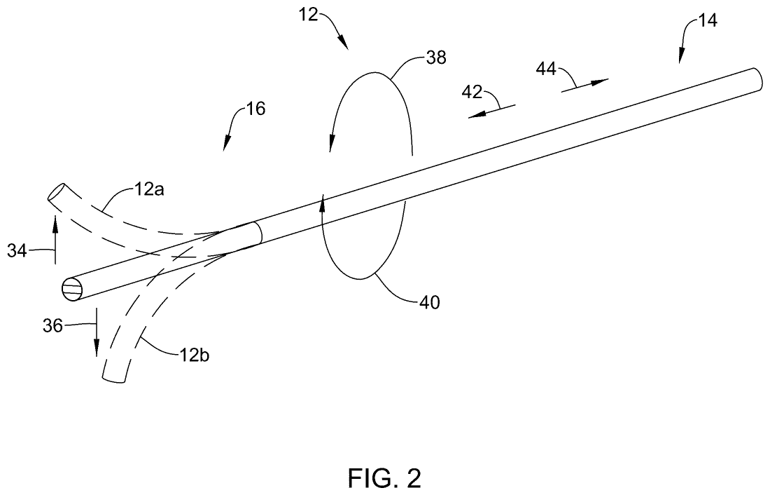

In some instances, the steerable elongate medical device 10, including the composite shaft 12, may be adapted to facilitate advancing through tortuous vasculature, including accessing side branches. FIG. 2 is a schematic view of the composite shaft 12, showing the degrees of freedom afforded by the steerable elongate medical device 10. The distal region 16 is adapted to articulate up and down. A shaft portion 12a is shown in phantom, showing how the distal region 16 is able to bend or curve in a first direction indicated by an arrow 34. A shaft portion 12b is shown in phantom, showing how the distal region 16 is able to bend or curved in a second direction indicated by an arrow 36. The composite shaft 12 is adapted to be rotated in either a clockwise direction or a counter clockwise direction, as indicated by arrows 38 and 40, respectively. The composite shaft 12 is adapted to be pushed in a direction indicated by an arrow 42 and to be pulled in a direction indicated by an arrow 44. By combining these motions, the composite shaft 12 may be navigated through a tortuous vasculature and even into a side branch, for example. The composite shaft 12 may be steered in any direction by rotating the composite shaft 12 and then actuating the distal region 16 in either the first direction indicated by the arrow 34 or the second direction indicated by the arrow 36.

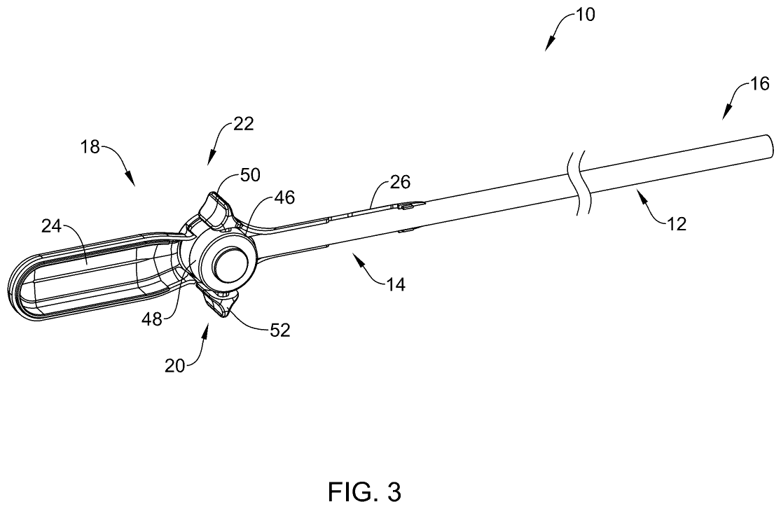

FIG. 3 is a perspective view of the illustrative steerable elongate medical device 10, with part of the housing 20 removed. It can be seen that the proximal region 14 of the composite shaft 12 extends well into the distal region 26 of the housing 20. In some instances, the composite shaft 12 has a proximal end 46 that is positioned proximate the knob 22. As will be discussed, positioning the proximal end 46 of the composite shaft 12 proximate the knob 22 allows for selectively actuating the composite shaft 12 to bend or curve in the direction indicated by the arrow 34, to bend or curve in the direction indicated by the arrow 36, or to remain in a neutral configuration in which the composite shaft 12 may be curved as a result of the vasculature in which the steerable elongate medical device 10 is placed in, but will remain linear or substantially linear (defined as within ten percent of linear) if not otherwise constrained. The knob 22 includes a knob body 48. A first graspable member 50 extends radially outwardly from the knob body 48 in a first direction and a second graspable member 52 extends radially outwardly from the knob body 48 in a second, opposing direction. The first graspable member 50 and the second graspable member 52, in combination, permit a user to easily rotate the knob 22 in order to selectively actuate the composite shaft 12.

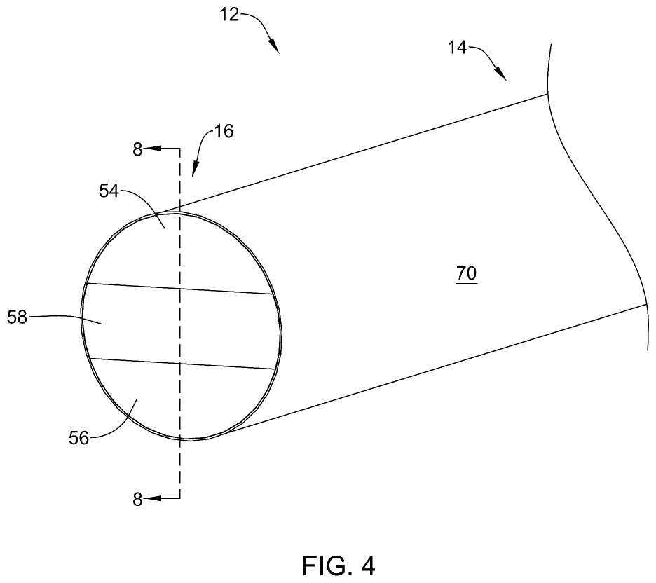

FIG. 4 is a schematic perspective view of a portion of the composite shaft 12. The composite shaft 12 includes a first shaft component 54 and a second shaft component 56. The first shaft component 54 is adapted to have a first or straight configuration below a transition temperature and a second or curved configuration above the transition temperature, the first shaft component 54 causing the composite shaft 12 to bend in the first direction when the first shaft component 54 is in its curved configuration. The first or straight configuration may correspond to a neutral configuration. As an example, in FIG. 2, the composite shaft 12 has bent or curved in the first direction indicated by the arrow 34 and the shaft portion 12a (shown in phantom). In some instances, the first shaft component 54 may be configured to move from the first or straight configuration to the second or curved configuration as a result of being electrically heated by running an electrical current through the first shaft component 54. In some instances, the first shaft component 54 may include a shape memory material. In some instances, the first shaft component 54 may have a remembered configuration corresponding to the second or curved configuration. In some instances, the first shaft component 54 may be made of nitinol.

The second shaft component 56 is adapted to have a first or straight configuration below the transition temperature and a second or curved configuration when above the transition temperature, the second shaft component 56 causing the composite shaft 12 to bend in the second direction when the second shaft component 56 is in its curved configuration. The first or straight configuration may correspond to a neutral configuration. As an example, in FIG. 2, the composite shaft 12 has bent or curved in the second direction indicated by the arrow 36 and the shaft portion 12b (shown in phantom). In some instances, the second shaft component 56 may be configured to move from the first or straight configuration to the second or curved configuration as a result of being electrically heated by running an electrical current through the second shaft component 56. In some instances, the second shaft component 56 may include a shape memory material. In some instances, the second shaft component 56 may have a remembered configuration corresponding to the second or curved configuration. In some instances, the second shaft component 56 may be made of nitinol.

In some instances, the transition temperature may correspond to a temperature that is greater than body temperature. The transition temperature may be above 37° C., for example. The transition temperature may be above 40° C., or above 42° C. This means that the composite shaft 12 may have a neutral configuration at lower temperatures, such as ambient temperature, or even at normal body temperature. When the first shaft component 54 is heated above the transition temperature, the first shaft component 54 will regain its remembered configuration, which causes the composite shaft 12 to curve in the first direction. When the second shaft component 56 is heated above the transition temperature, the second shaft component 56 will regain its remembered configuration, which causes the composite shaft 12 to curve in the second direction. In some instances, the first shaft component 54 and the second shaft component 56 may remain malleable when below the transition temperature. In some instances, the first shaft component 54 and the second shaft component 56 may each become super-elastic as a result of being electrically heated.

The composite shaft 12 includes an intervening member 58 that is disposed between the first shaft component 54 and the second shaft component 56. In some instances, the intervening member 58 may have insulative properties. In some instances, the intervening member 58 may be adapted to be electrically insulating, such that an electrical current flowing through one of the first shaft component 54 and the second shaft component 56 may not cross into the other of the first shaft component 54 and the second shaft component 56. In some instances, the intervening member 58 may be adapted to be thermally insulating, such that increased temperature within one of the first shaft component 54 and the second shaft component 56 does not warm up the other of the first shaft component 54 and the second shaft component 56. The intervening member 58 may be formed of a variety of different polymeric materials, for example. The intervening member 58 may include or be made of PTFE (polytetrafluoroethylene), Pebax®, PVC (polyvinyl chloride) or any type of foam.

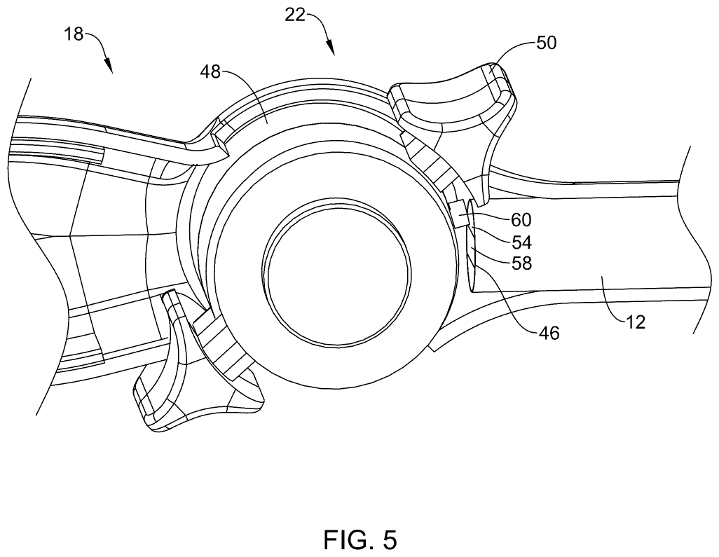

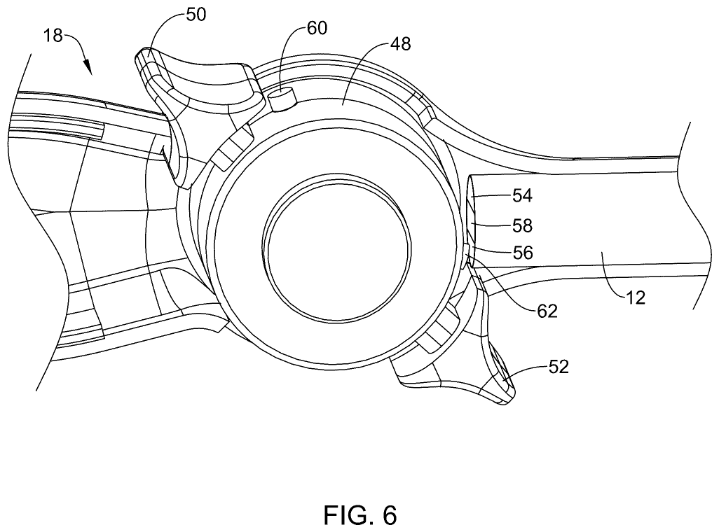

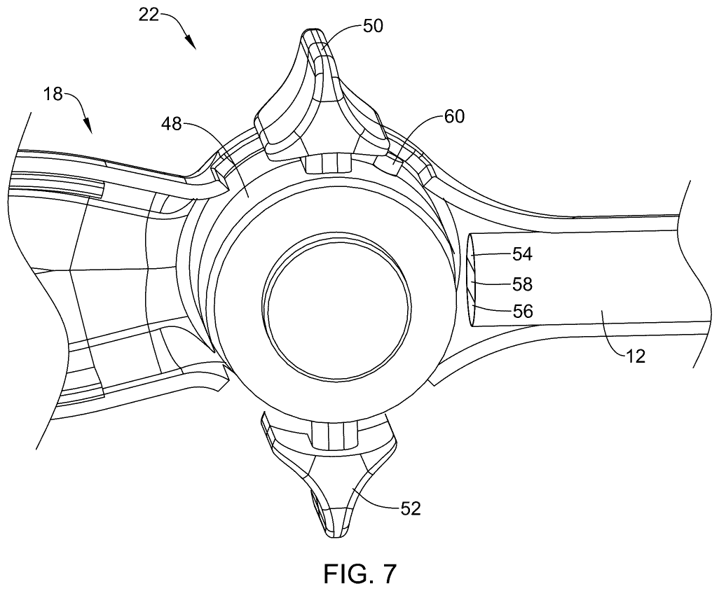

In some instances, the knob 22 may be rotatable between a first position in which an electrical current is provided to the first shaft component 54, a second position in which an electrical current is provided to the second shaft component 56 and a third position in which no electrical current is provided. In some instances, the handle 18 may be considered as being selectable between the first position, the second position and the third position. FIG. 5 shows the knob 22 in the first position, FIG. 6 shows the knob 22 in the second position and FIG. 7 shows the knob 22 in the third position. In some instances, the knob 22 may be freely movable between the first position, the second position and the third position, and may be adapted to remain in whichever position the knob 22 is turned to. In some instances, the knob 22 may be biased to the third position, for example, and will only remain in the first position or the second position for as long as someone is holding the knob 22 in the first position or the second position, respectively.

In FIG. 5, the knob 22 has been rotated such that the first graspable member 50 has moved distally and is positioned closer to the composite shaft 12 and the second graspable member 52 has moved proximally and is positioned farther away from the composite shaft 12. In some instances, the relative positions of the first graspable member 50 and the second graspable member 52 provide a visual indication of the position of the knob 22, and thus an indication of which shaft component (if any) is being heated. The knob 22 includes a first electrical contact 60 that rotates with the knob 22. In some instances, the first electrical contact 60 is biased in an outward direction, such as by a spring (not shown). In the illustrated position, the first electrical contact 60 is in electrical contact with the first shaft component 54. As a result, electrical current is provided through the first shaft component 54, and the electrical resistance of the first shaft component 54 causes the first shaft component 54 to increase in temperature. As the temperature of the first shaft component 54 increases above the transition temperature, the first shaft component 54 will regain its remembered shape and will curve or bend. This will cause the composite shaft 12 to curve or bend in the first direction, as indicated by the arrow 34 (FIG. 2).

In FIG. 6, the knob 22 has been rotated such that the first graspable member 50 has moved proximally and is positioned farther from the composite shaft 12 and the second graspable member 52 has moved distally and is positioned closer to from the composite shaft 12. In some instances, the relative positions of the first graspable member 50 and the second graspable member 52 provide a visual indication of the position of the knob 22, and thus an indication of which shaft component (if any) is being heated. The knob 22 includes a second electrical contact 62 that rotates with the knob 22. In some instances, the second electrical contact 62 is biased in an outward direction, such as by a spring (not shown). In the illustrated position, the second electrical contact 62 is in electrical contact with the second shaft component 56. As a result, electrical current is provided through the second shaft component 56, and the electrical resistance of the second shaft component 56 causes the second shaft component 56 to increase in temperature. As the temperature of the second shaft component 56 increases above the transition temperature, the second shaft component 56 will regain its remembered shape and will curve or bend. This will cause the composite shaft 12 to curve or bend in the second direction, as indicated by the arrow 36 (FIG. 2).

In FIG. 7, the knob 22 has been rotated such that the first graspable member 50 and the second graspable member 52 are disposed in a vertical position. In some instances, the relative positions of the first graspable member 50 and the second graspable member 52 provide a visual indication of the position of the knob 22, and thus an indication of which shaft component (if any) is being heated. With the knob 22 in this position, the first electrical contact 60 is held away from contact with the first shaft component 54 and the second electrical contact 62 is held away from contact with the second shaft component 56. As a result, no electrical current flows through either of the first shaft component 54 or the second shaft component 56. This means that the first shaft component 54 and the second shaft component 56 will both remain in their first or linear configurations, and the composite shaft 12 will also retain a straight configuration, apart from any incidental curves of the composite shaft 12 as a result of being constrained by vessel walls impacting the composite shaft 12.

With brief reference to FIG. 4, the composite shaft 12 includes an outer layer 70. In some instances, the outer layer 70 may be a polymeric extrusion that electrically and thermally insulates the composite shaft 12. In some instances, the outer layer 70 may be formed of a Pebax or PTFE extrusion. In some instances, the outer layer 70 may provide additional shaft stiffness and as such may include one or more reinforcing layers such as a coil or a braid, for example. In some cases, the outer layer 70 may include several distinct layers, such as two or more polymeric insulating layers sandwiching a conductive layer that extends to the distal region 16 of the composite shaft 12 in order to provide an electrical conductor that is in electrical contact with the distal portions of the first shaft component 54 and the second shaft component 56 in order to complete an electrical circuit such that electrical current is able to flow.

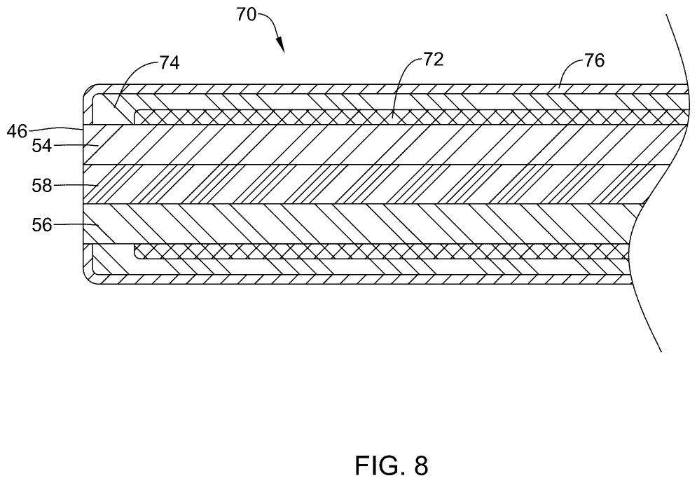

FIG. 8 is a cross-sectional view taken along the line 8-8 of FIG. 4. As can be seen in FIG. 8, a first insulating layer 72 is disposed directly over the first shaft component 54. A conductive layer 74 is disposed over the first insulating layer 72. In some instances, the conductive layer 74 may extend farther distally than the first insulating layer 72, and as a result the conductive layer 74 may make electrical contact with the first shaft component 54 (as shown) as well as the second shaft component 56, and thus the conductive layer 74 may function as a return path for the electrical current selectively provided to the first shaft component 54 and the second shaft component 56. A second insulating layer 76 may be disposed over the conductive layer 74 in order to electrically isolate the conductive layer 74. While a three layer construction is shown, it will be appreciated that this is merely illustrative, and the composite shaft 12 may include any number of insulating polymeric layers. The composite shaft 12 may include alternative examples of an electrical conductor extending to the distal end 46 of the composite shaft 12. As an example, the composite shaft 12 may simply include a conductive wire extending the length of the composite shaft 12.

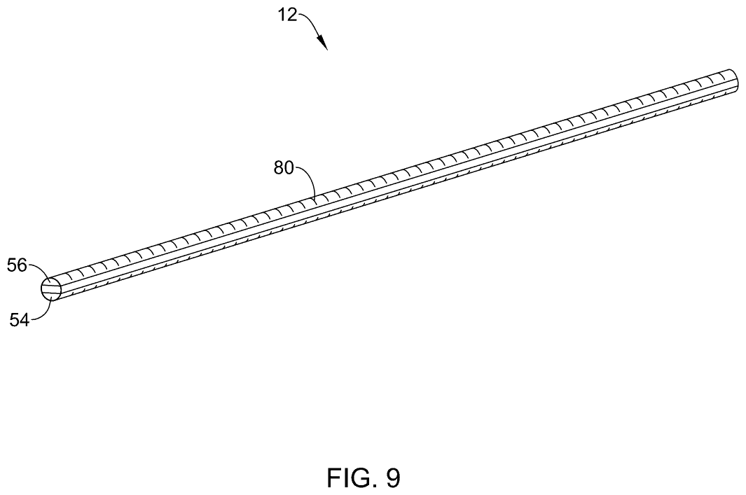

In some instances, the first shaft component 54 and/or the second shaft component 56 may include a pattern of cuts formed within the first shaft component 54 and/or the second shaft component 56. In some instances, forming a pattern of cuts may make each of the first shaft component 54 and/or the second shaft component 56 more flexible. When one of the first shaft component 54 and the second shaft component 56 is heated, the heated shaft component transitions to its remembered shape. The non-heated shaft component remains malleable, and thus the heated shaft component is able to overcome the shape of the non-heated component, allowing the composite shaft 12 to bend or curve. FIG. 9 shows the composite shaft 12 with a pattern of cuts 80 formed within an outer surface of the second shaft component 56. The pattern of cuts 80 may improve articulation of the first shaft component 54 and the second shaft component 56. This is just an example, and any of a variety of different cutting patterns may be used. In some instances, the composite shaft 12 may be adapted to bend equally in the first direction and in the second direction. In some instances, the composite shaft 12 may be adapted to preferentially bend in one direction over another direction. The relative cutting patterns used on each of the first shaft component 54 and the second shaft component 56 may reflect the desired bending characteristics of the composite shaft 12.

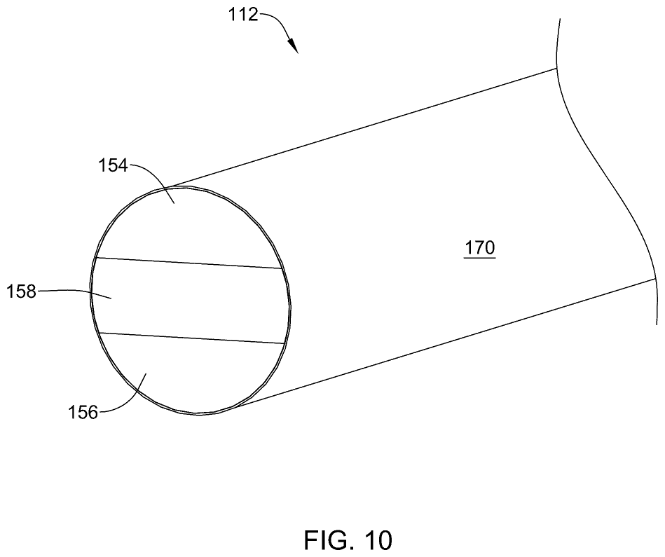

FIG. 10 is a schematic perspective view of a portion of a composite shaft 112. The composite shaft 112 includes a first shaft component 154 and a second shaft component 156. The first shaft component 154 is adapted to have a first or straight configuration below a transition temperature and a second or curved configuration above the transition temperature, the first shaft component 154 causing the composite shaft 112 to bend in the first direction when the first shaft component 154 is in its curved configuration. The first or straight configuration may correspond to a neutral configuration. In some instances, the first shaft component 54 may be configured to move from the first or straight configuration to the second or curved configuration as a result of being heated. In some instances, the first shaft component 154 may include a shape memory material. In some instances, the first shaft component 154 may have a remembered configuration corresponding to the second or curved configuration. In some instances, the first shaft component 154 may be made of nitinol.

The second shaft component 156 is adapted to have a first or straight configuration below the transition temperature and a second or curved configuration when above the transition temperature, the second shaft component 156 causing the composite shaft 112 to bend in the second direction when the second shaft component 156 is in its curved configuration. The first or straight configuration may correspond to a neutral configuration. In some instances, the second shaft component 156 may be configured to move from the first or straight configuration to the second or curved configuration as a result of being heated. In some instances, the second shaft component 156 may include a shape memory material. In some instances, the second shaft component 156 may have a remembered configuration corresponding to the second or curved configuration. In some instances, the second shaft component 156 may be made of nitinol.

In some instances, the transition temperature may correspond to a temperature that is greater than body temperature. The transition temperature may be above 37° C., for example. The transition temperature may be above 40° C., or above 42° C. This means that the composite shaft 112 may have a neutral configuration at lower temperatures, such as ambient temperature, or even at normal body temperature. When the first shaft component 154 is heated above the transition temperature, the first shaft component 154 will regain its remembered configuration, which causes the composite shaft 112 to curve in the first direction. When the second shaft component 156 is heated above the transition temperature, the second shaft component 156 will regain its remembered configuration, which causes the composite shaft 112 to curve in the second direction. In some instances, the first shaft component 154 and the second shaft component 156 may remain malleable when below the transition temperature. In some instances, the first shaft component 154 and the second shaft component 156 may each become super-elastic as a result of being heated.

The composite shaft 112 includes an intervening member 158 that is disposed between the first shaft component 154 and the second shaft component 156. In some instances, the intervening member 158 may be a thermoelectric circuit that is adapted to selectively heat one of the first shaft component 154 and the second shaft component 156. In some cases, the thermoelectric circuit may include materials having different Seebeck coefficients (p-doped and n-doped semiconductors), configured as a thermoelectric generator. The composite shaft 112 includes an outer layer 170 that may include a polymeric insulating layer, for example.

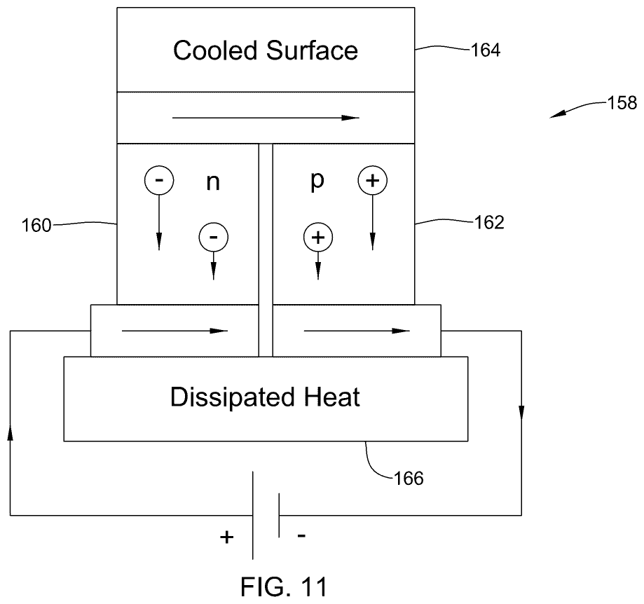

FIG. 11 is a schematic diagram showing the thermoelectric circuit 158. The thermoelectric circuit 158 includes an n-doped semiconductor 160 and a p-doped semiconductor 162. Running a current through will result in a cooled surface 164 and a heated surface 166. Depending on which direction the current is flowing, which can be controlled, it is possible to reverse which surface is heated and which surface is cooled. If the cooled surface 164 corresponds to the first shaft component 154 and the heated surface 166 corresponds to the second shaft component 156, the second shaft component 156 will heat up and bend, causing the composite shaft 112 to bend a direction indicated by the remembered shape of the second shaft component 156. Alternatively, if the cooled surface 164 corresponds to the second shaft component 156 and the heated surface 166 corresponds to the first shaft component 156154 the first shaft component 154 will heat up and bend, causing the composite shaft 112 to bend a direction indicated by the remembered shape of the first shaft component 154.

The materials that can be used for the various components of the medical devices described herein may include those commonly associated with medical devices. The medical devices described herein, as well as individual components thereof, be made from a metal, metal alloy, polymer (some examples of which are disclosed below), a metal-polymer composite, ceramics, combinations thereof, and the like, or other suitable material. Some examples of suitable metals and metal alloys include stainless steel, such as 304, 304V, 304L, 316, 316LV, 303, 410 and 416 stainless steel; nickel-titanium alloys such as linear-elastic and/or super-elastic nitinol; cobalt-nickel-chromium-molybdenum alloys (e.g., UNS: R30035 such as MP35N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, and the like; cobalt-chromium alloys; cobalt-chromium-molybdenum alloys (e.g., UNS: R30003 such as ELGILOY®, PHYNOX®, and the like); brasses such as C360, C260, C280, C693, C768, C87850 and the like; platinum enriched stainless steel; combinations thereof; and the like; or any other suitable material.

The medical devices described herein, as well as portions and components thereof, may be made of the same material along its length, or in some embodiments, can include portions or sections made of different materials. In some embodiments, materials may be chosen to impart varying flexibility and stiffness characteristics to different portions. For example, different portions of a component, such as a proximal section and a distal section, may be formed of different materials, for example, materials having different moduli of elasticity, resulting in a difference in flexibility. In some embodiments, the material used to construct a proximal section may be relatively stiff for pushability and torqueability, and the material used to construct a distal section may be relatively flexible by comparison for better lateral trackability and steerability. For example, a proximal section may be formed of straightened 304v stainless steel wire or ribbon and a distal section may be formed of a straightened super elastic or linear elastic alloy, for example a nickel-titanium alloy wire or ribbon.

In embodiments where different portions of the medical devices described herein are made of different materials, the different portions can be connected using a suitable connecting technique and/or with a connector. For example, the different portions may be connected using welding (including laser welding), soldering, brazing, adhesive, or the like, or combinations thereof. These techniques can be utilized regardless of whether or not a connector is utilized. An example of a connector is a structure such as a hypotube or a coiled wire which has an inside diameter sized appropriately to receive and connect to the ends of the proximal portion and the distal portion.

A sheath or covering (not shown) may be disposed over portions or all of the medical devices described herein. In other embodiments, however, such a sheath or covering may be absent. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP. The sheath may include braided wire along some or all of the length of the shaft, for example.

In some embodiments, the exterior surface of the medical devices described herein may be sandblasted, beadblasted, sodium bicarbonate-blasted, electropolished, etc. In these as well as in some other embodiments, a coating, for example a lubricious, a hydrophilic, a protective, or other type of coating may be applied over portions or all of the medical devices described herein. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Having thus described several illustrative embodiments of the present disclosure, those of skill in the art will readily appreciate that yet other embodiments may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A steerable elongate medical device, comprising:
a composite shaft extending from a proximal region to a distal region, the distal region adapted to selectively bend in a first direction or an opposing second direction, the composite shaft including:
a first shaft component having a straight configuration below a transition temperature and a curved configuration above the transition temperature, the first shaft component causing the composite shaft to bend in the first direction when the first shaft component is in its curved configuration;
a second shaft component having a straight configuration below the transition temperature and a curved configuration when above the transition temperature, the second shaft component causing the composite shaft to bend in the second direction when the second shaft component is in its curved configuration; and
an intervening member separating the first shaft component and the second shaft component; and
a handle removably secured to the proximal region, the handle including:
a housing adapted to be removably secured to the proximal region of the composite shaft; and
a knob rotatably secured relative to the housing, the knob including:
a knob body;
a first electrical contact disposed on the knob body and adapted to contact the first shaft component when the knob is rotated to a first position; and
a second electrical contact disposed on the knob body and adapted to contact the second shaft component when the knob is rotated to a second position.

2. The steerable elongate medical device of claim 1, wherein:
the first shaft component is adapted to increase in temperature when an electrical current passes through the first shaft component; and
the second shaft component is adapted to increase in temperature when an electrical current passes through the second shaft component.

3. The steerable elongate medical device of claim 1, wherein the first shaft component and the second shaft component each comprise a shape memory metal.

4. The steerable elongate medical device of claim 1, wherein the first shaft component and the second shaft component each comprise nitinol.

5. The steerable elongate medical device of claim 2, wherein:
the first electrical contact is adapted to make electrical contact with the first shaft component when the knob is rotated to the first position; and
the second electrical contact is adapted to make electrical contact with the second shaft component when the knob is rotated to the second position.

6. The steerable elongate medical device of claim 1, wherein the knob is adapted to be rotated to a neutral position in between the first position and the second position in which neither of the first electrical contact or the second electrical contact make electrical contact with either the first shaft component or the second shaft component.

7. The steerable elongate medical device of claim 2, wherein the knob further comprises a power connector that is electrically coupled with the first electrical contact and the second electrical contact.

8. The steerable elongate medical device of claim 2, further comprising a conductive member in electrical contact with a distal region of the first shaft component and a distal region of the second shaft component in order to complete a circuit.

9. The steerable elongate medical device of claim 1, wherein the transition temperature is above 37° C.

10. The steerable elongate medical device of claim 1, wherein the intervening member comprises an insulating member.

11. The steerable elongate medical device of claim 1, wherein the intervening member comprises a thermoelectric circuit adapted to selectively heat one of the first shaft component and the second shaft component.

12. The steerable elongate medical device of claim 1, wherein the first shaft component includes a plurality of cuts formed within the first shaft component in order to enhance articulation of the first shaft component.

13. The steerable elongate medical device of claim 1, wherein the second shaft component includes a plurality of cuts formed within the second shaft component in order to enhance articulation of the second shaft component.

14. A steerable guidewire, comprising:
a composite shaft adapted to selectively bend in a first direction or an opposing second direction, the composite shaft including:
a first shaft component movable between a first configuration and a second configuration, the first shaft component adapted to move into the second configuration when electrically heated; and
a second shaft component movable between a first configuration and a second configuration, the second shaft component adapted to move into the second configuration when electrically heated; and
a handle adapted to be removably secured to the composite shaft, the handle selectable between:
a first position in which an electrical current is provided to the first shaft component;
a second position in which an electrical current is provided to the second shaft component; and
a third position in which no electrical current is provided.

15. The steerable guidewire of claim 14, wherein the first shaft component and the second shaft component each comprise a shape memory metal.

16. The steerable guidewire of claim 14, wherein the first shaft component and the second shaft component each comprise nitinol.

17. The steerable guidewire of claim 14, wherein the first configuration of the first shaft component and the first configuration of the second shaft component each correspond to a straight configuration of the composite shaft.

18. The steerable guidewire of claim 14, wherein:
the first shaft component causes the composite shaft to bend in the first direction when the first shaft component is electrically heated and the second shaft component is not electrically heated; and
the second shaft component causes the composite shaft to bend in the second direction when the second direction is electrically heated and the first shaft component is not electrically heated.

19. The steerable guidewire of claim 14, wherein the composite shaft has a neutral configuration when neither the first shaft component nor the second shaft component are electrically heated.

20. A steerable guidewire, comprising:
a composite shaft adapted to selectively bend in a first direction or an opposing second direction, the composite shaft including:
a first shaft component movable between a neutral configuration and a curved configuration, the first shaft component adapted to move into the curved configuration when electrically heated, the first shaft component comprising nitinol; and
a second shaft component movable between a neutral configuration and a curved configuration, the second shaft component adapted to move into the curved configuration when electrically heated, the second shaft component comprising nitinol;
wherein the composite shaft is adapted to bend in the first direction when the first shaft component is in its curved configuration and the second shaft component is in its neutral configuration;
wherein the composite shaft is adapted to bend in the opposing second direction when the first shaft component is in its neutral configuration and the second shaft component is in its curved configuration; and
wherein the composite shaft is adapted to remain in a neutral configuration when the first shaft component is in its neutral configuration and the second shaft component is in its neutral configuration.
